(19) United States
(12) Patent Application Publication
(10) Pub. No.: US 2003/0093038 A1
(43) Pub. Date: May 15, 2003

(54) Needle retraction type safety syringe

(76) Inventor: Kun-Feng Chiang, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/987,510

(22) Filed: Nov. 15, 2001

(51) Int. Cl. A61M 5/00

(52) U.S. Cl. 604/240

(57) ABSTRACT

The present invention provides a safety syringe. A second positioning means is disposed on the inner wall of a barrel. The front end of a passive needle retraction element is engaged with a needle. An active needle retraction element having a rear section larger than the bore of the barrel exactly contracts and clamps the inner end of a push rod when implanted into the barrel. The passive needle retraction element after engaged with the active needle retraction element can be forced to separate from the positioning restriction of the second positioning means so that the needle can be retracted into the barrel by pulling back the push rod. When the rear section of the active needle retraction element emerges out of the rear end of the barrel, its contraction state will restore to the expansion state to release clamping to the push rod.

### FIELD OF THE INVENTION

The present invention relates to a needle retraction type safety syringe and, more particularly, to a safety syringe, wherein a needle or a mechanism to which the needle pertaining is engaged with a push rod so that the needle can be retracted after the end of injection stroke. The needle can be retracted into a barrel by pulling back the push rod and the pushed rod can be released automatically at the end of retraction stroke to keep the needle inside the barrel for reducing needlestick injuries among health-care workers.

### BACKGROUND OF THE INVENTION

Because cases about infection through blood have usually been heard, it is regulated that a syringe (or called a needle) penetrating the human body to be contaminated with blood can only be used once.

As shown in FIG. 1, a conventional syringe (needle) comprises a push rod 3 having a plunger 31 at an inner end thereof, a barrel 4, and a needle 5. The front end of the barrel 4 is telescoped with the tail end of the needle 5. The push rod 3 is implanted from the rear end of the barrel 4. Because the needle 5 after usage is still kept outside, disposal people usually carelessly touch the needle 5 to penetrate their skins, resulting in uncalled infection. Although there has been a needle sheath (not shown) sleeved outside the needle 5 of a contaminated needle to avoid careless touch, in addition to inconvenience, the needle sheath sleeved on the needle 5 usually falls off to cause trouble in safety.

Accordingly, the present invention aims to propose a safety syringe to resolve the problems in the prior art.

### SUMMARY OF THE INVENTION

The primary object of the present invention is to provide a needle retraction type safety syringe, wherein a needle or a mechanism to which the needle pertaining is exactly engaged with a push rod at the end of injection stroke so that it is only necessary to pull back the push rod to retract the needle into a barrel. The operation is very simple. Additionally, the engagement state is released once the push rod is exposed out of the barrel, and the push rod and barrel are separated off when the needle is retracted, hence avoiding the situation of a too long syringe.

To achieve the above object, the present invention provides a needle retraction type safety syringe, wherein the dead end of the injection stroke of a push rod is exactly situated at the front end of a barrel, and a passive needle retraction element temporarily limited by a second positioning means on the inner wall of the barrel is disposed at the front end of the barrel. The front end of the passive needle retraction element is engaged with a needle. An active needle retraction element having a resilient and expanding rear section larger than the bore of the barrel is designed to exactly contract and clamp the inner end of the push rod when implanted into the barrel. The front section of the active needle retraction element can be engaged with the passive needle retraction element. The passive needle retraction element after engaged with the active needle retraction element is forced to separate from positioning restriction of the second positioning means so that the needle can be retracted into the barrel by pulling back the push rod. Moreover, when the rear section of the active needle retraction element is exposed out of the rear end of the barrel, its contraction state restores to the expansion state so that clamping to the push rod is released.

The various objects and advantages of the present invention will be more readily understood from the following detailed description when read in conjunction with the appended drawings, in which:

### BRIEF DESCRIPTION OF THE DRAWINGS

FIG. 1 is a plan view of a prior art syringe;

FIG. 2 is an exploded drawing of the present invention;

FIG. 3 is a perspective view of the present invention;

FIG. 4 is a cross-sectional view of the present invention;

FIG. 4A is a detail drawing of part A shown in FIG. 4;

FIG. 4B is a detail drawing of part B shown in FIG. 4;

FIG. 5 is a cross-sectional view of the present invention after injection;

FIG. 5A is a detail drawing of FIG. 5;

FIG. 6 is a cross-sectional view of the present invention after the needle is retracted;

FIG. 7 is a cross-sectional view of another embodiment of the present invention;

FIG. 7A is a detail drawing of FIG. 7; and

FIG. 8 is a detail drawing of yet another embodiment of the present invention.

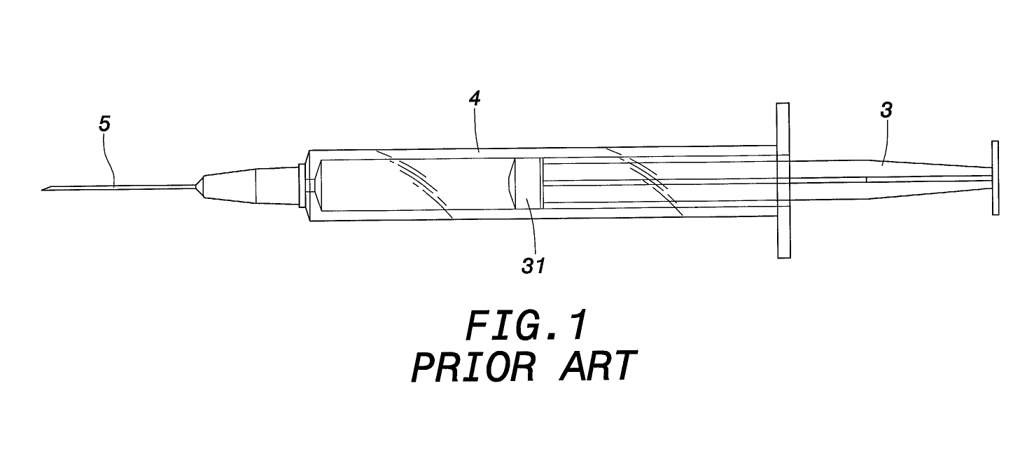

### DETAILED DESCRIPTION OF THE PREFERRED EMBODIMENTS

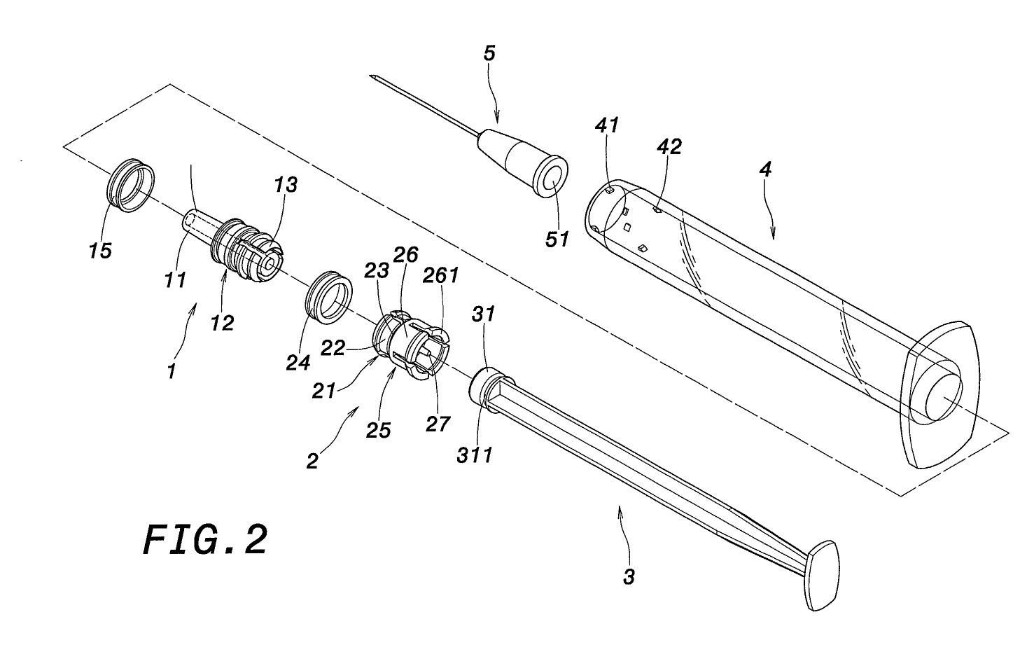

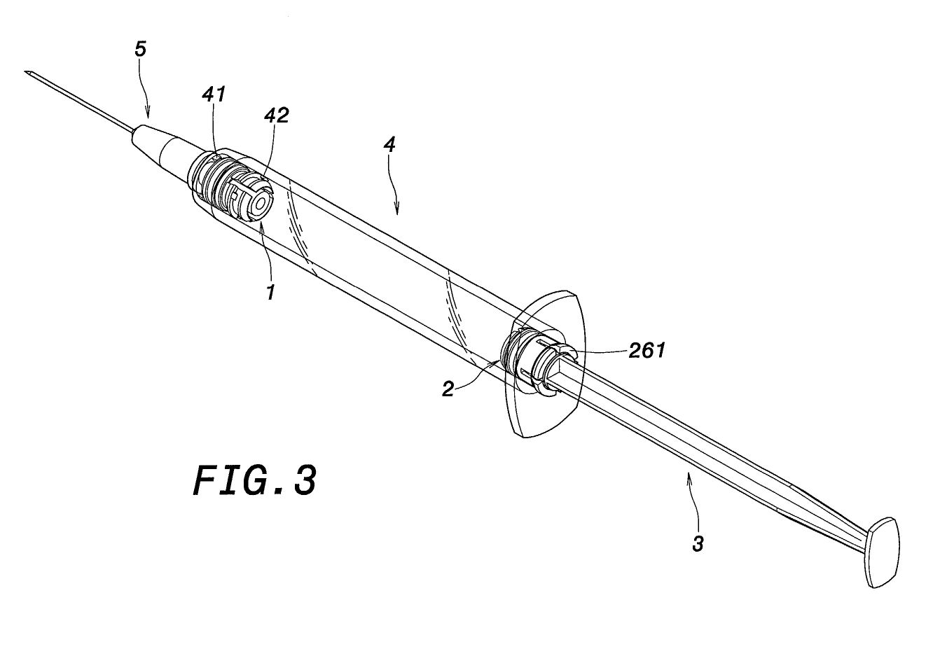

As shown in FIGS. 2 and 3, the present invention provides a needle retraction type safety syringe comprising a passive needle retraction element 1, an active needle retraction element 2, a push rod 3, a barrel 4, and a needle 5.

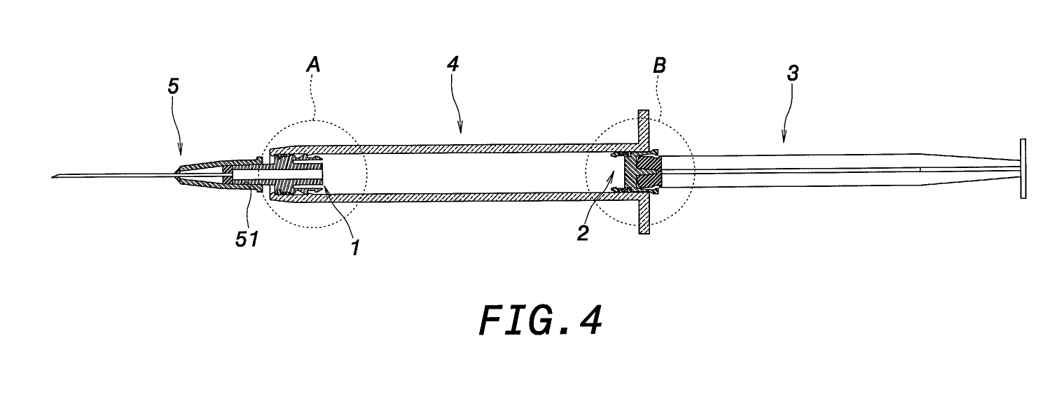

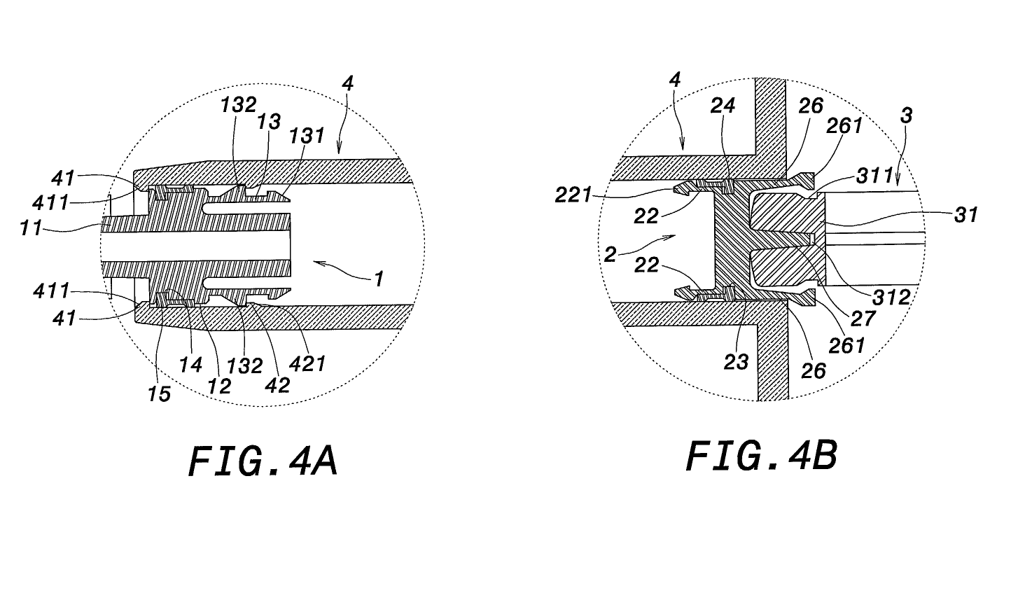

Please refer to FIGS. 4, 4A, and 4B, an implantation hole 51 is axially disposed at the tail end of the needle 5. The barrel 4 is hollow. A first positioning means 41 and a second positioning means 42 having a plurality of protuberances are disposed on the inner wall of the front end of the barrel 4. The protuberances of the first and second positioning means 41 and 42 are preferably to be staggered. As shown in FIG. 4A, opposite inner sides of the protuberances of the first and second positioning means 41 and 42 are flat, while opposite outer sides thereof form outer slanting edges 411 and inner slanting edges 421 of inclined shape.

Moreover, the passive needle retraction element 1 comprises a collar 11 and a held body 12. The held body 12 sheathes the outer edge of the collar 11. The collar 11 protrudes out of the front end of the held body 12 to be sleeved into the implantation hole 51 of the needle 5. Please also refer to FIG. 4A, a plurality of fastening claws 13 are annularly disposed and extended backwards from the held body 12. The rear edge of each of the fastening claw 13 forms a raised second fastening body 131. The outer edge of each of the fastening claws 13 forms an abutting edge 132 capable of abutting against the second positioning means 42. The front edge of the held body 12 abuts against the first positioning means 41. In order not to let infusion leak out, an embedding trench 14 is annularly disposed near the front edge of the held body 12 to embed a gasket 15.

Please also refer to FIG. 4B, the active needle retraction element 2 comprises a front holding body 21 and a rear holding body 25 back to back. A plurality of fastening claws 22 and 26 are annularly disposed and respectively extended forwards and backwards from the front and rear holding bodies 21 and 25 respectively pertaining to the front and rear sections of the active needle retraction element 2. The front edge of each of the fastening claws 22 of the front holding body 21 forms a first fastening body 221 projecting inwards. The rear edge of each of the fastening claws 26 of the rear holding body 25 forms an expansion flange 261 larger than the bore of the barrel 4 so that the expansion flange 261 is limited to contract when the active needle retraction element 2 is implanted into the barrel 4. The bore of the rear opening enclosed by the fastening claws 26 will thus be shrunk due to contraction. Also, in order not to let infusion leak out, an embedding trench 23 is annularly disposed between the front and rear holding bodies 21 and 25 to embed a gasket 24.

An annular groove 311 corresponding to the expansion flange 261 is disposed at the outer edge of the plunger 31 at the inner end of the push rod 3 so that the expansion flange 261 of the active needle retraction element 2 can clamp the push rod 3.

Please refer to FIGS. 3, 4, 4A, and 4B, the passive needle retraction element 1 can be successfully implanted at the front end of the barrel 4 by means of the edges 411 of the first positioning means 41. The passive needle retraction element 1 can be positioned between the first and second positioning means 41 and 42 because the abutting edge 132 of the passive needle retraction element 1 abuts against the flat plane of the second positioning means 42. Because the flat front edge of the held body 12 exactly abuts against the corresponding flat plane of the first positioning means 41, the passive needle retraction element 1 cannot be retreated contrariwise.

The needle 5 is engaged at the front end of the barrel 4 by implanting the collar 11 of the passive needle retraction element 1 into the implantation hole 51 thereof.

The push rod 3 having the active needle retraction element 2 at the inner end thereof is inserted into the barrel 4 from the rear end of the barrel 4. When the push rod 3 is initially inserted, the expansion flange 261 of the active needle retraction element 2 has not been completely inserted into the barrel 4 yet so that the expansion flange 261 has not clamped the push rod 3 yet.

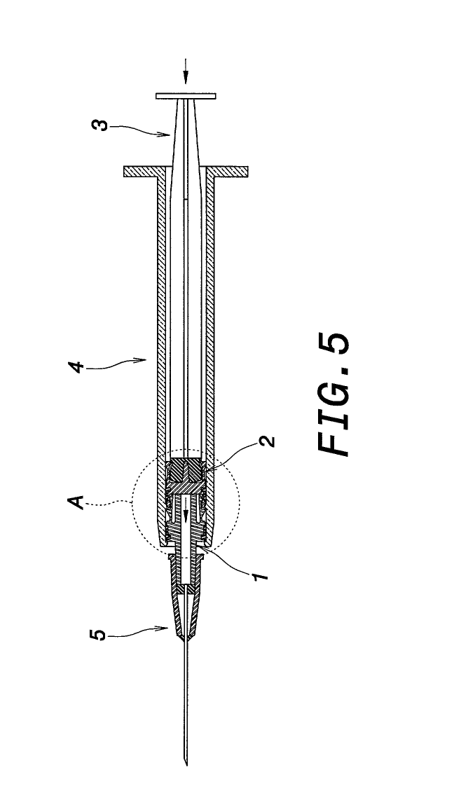

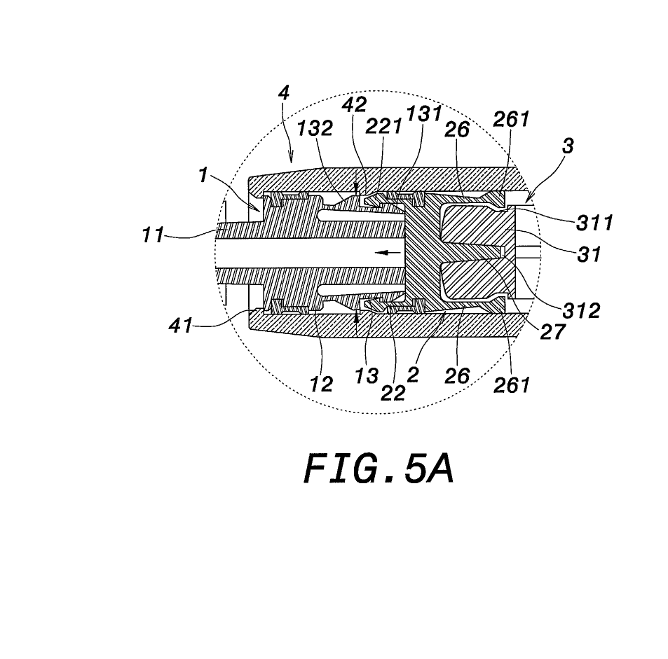

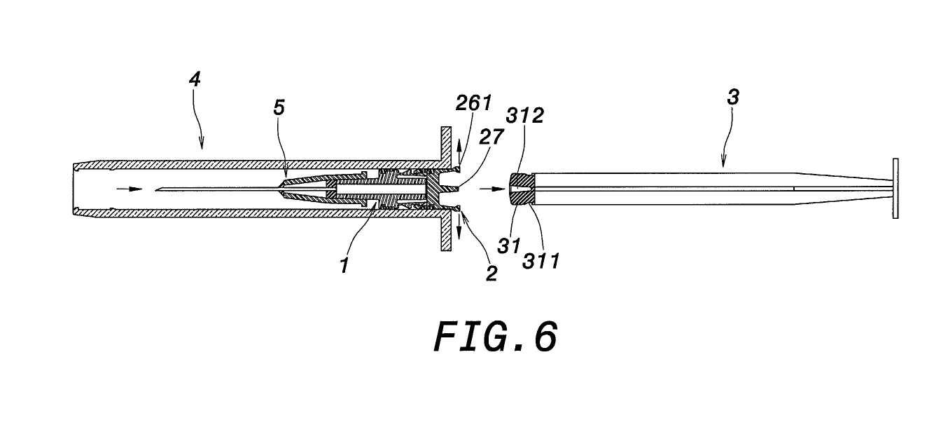

FIG. 5 is a cross-sectional view showing that the push rod 3 has completed its injection action and the injection stroke has arrived the dead end. FIG. 5A shows an enlarged cross-sectional view of the engaged active and passive needle retraction elements 2 and 1 shown in FIG. 5. When the push rod 3 takes the active needle retraction element 2 to continually move inwards, because the expansion flange 26 having an expansion diameter larger than the bore of the barrel 4 has already been received into the barrel 4 to contract, the contracted expansion flange 261 can be exactly embedded into the annular groove 311 of the plunger 31 of the push rod 3 so that the expansion flange 261 can clamp the inner end of the push rod 3. Because the diameter of the fastening claws 22 of the active needle retraction element 2 is slightly larger than that of the fastening claws 13 of the passive needle retraction element 1, when the push rod 3 has been pushed to the end of injection stroke, the second fastening bodies 131 at the rear edges of the fastening claws 13 of the passive needle retraction element 1 will be pressed inwards by the first fastening bodies 221 at the front edges of the fastening claws 22 of the active needle retraction element 2, generating resilient contraction of the fastening claws 13. When the rear end edges of the second fastening bodies 131 abut against the front wall of the active needle retraction element 2, the inwards projecting first fastening bodies 221 are exactly retained and engaged with the outwards projecting second fastening bodies 131, thereby joining the active and passive needle retraction elements 2 and 1. Through the fastening claws 22 of the active needle retraction element 2 pressing the fastening claws 13 of the passive needle retraction element 1, the abutting edges 132 at the outer edges of the fastening claws 13 can be jointly moved by contraction of the fastening claws 13 so that the abutting state of the abutting edges 132 originally abutting against the second positioning means 42 can be exactly released. The abutting edges 132 can thus be separated from the limit of the second positioning means 42. At this time, it is only necessary to pull back the push rod 3 to pull the passive needle retraction element 1 along with the needle 5 sleeved on the passive needle retraction element 1. When the needle retraction stroke of the push rod 3 has arrived the rear end, because the expansion flange 261 of the active needle retraction element 2 has already protruded out of the range of the barrel 4, it will resiliently restore to the expansion state from the original contraction state. The expansion flange 261 is thus released from the clamped state in the annular groove 311 so that the push rod 3 can be removed naturally. FIG. 6 is a view showing the action of easily separating the push rod 3 from the active needle retraction element 2. At this time, the needle 5 has already been retracted into the barrel 4 to ensure safety. Furthermore, because the expansion flange 261 expands resiliently, and the gaskets 24 and 15 annularly disposed at the active and passive needle retraction elements 2 and 1 abut against the inner wall of the barrel 4 simultaneously, the active needle retraction element 2 will not be easily moved so that the needle 5 will not emerge.

As shown in FIGS. 2 and 4B, in order that the push rod 3 can directly take the active needle retraction element 2 to be implanted into the barrel 4, an (or a plurality of) axially extending boss 27 is disposed at the rear end of the rear holding body 25 of the active needle retraction element 2, and a (or a plurality of) corresponding insertion hole 312 is formed at the corresponding end of the plunger 31 of the push rod 3 so that the active needle retraction element 2 can be roughly positioned at the inner end of the push rod 3.

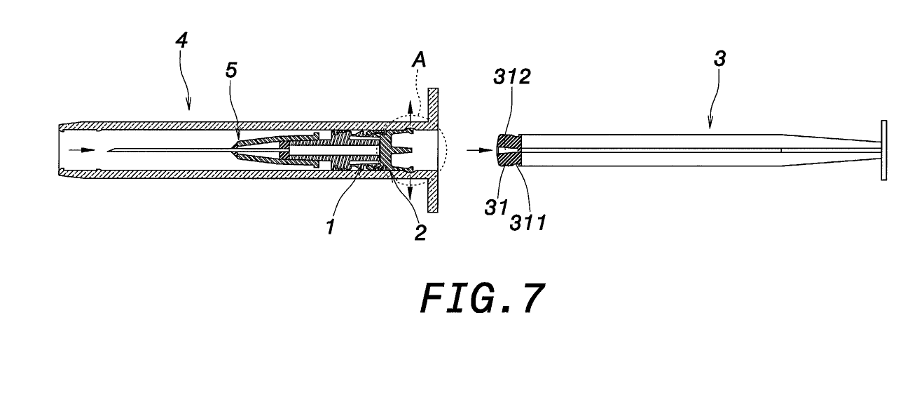

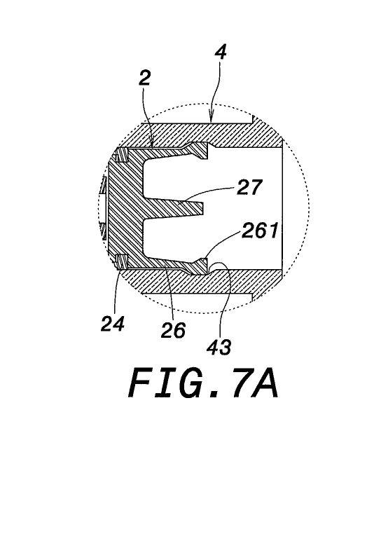

As shown in FIGS. 7 and 7A, a depressed concave edge 43 corresponding to the expansion flange 261 is disposed on the inner wall near the rear end of the barrel 4 so that the expansion flange 261 will immediately fall into the depressed concave edge 43 once passing therethrough when the push rod 3 along with the active and passive needle retraction elements 2 and 1 and the needle 5 is pulled back. The originally contracted expansion flange 261 will thus restore to the expansion state to release its clamping to the inner end of the push rod 3 so that the push rod 3 can be separated from the needle 5. Moreover, the length of the barrel 4 already receiving the needle 5 therein will not be too long to affect the littering process.

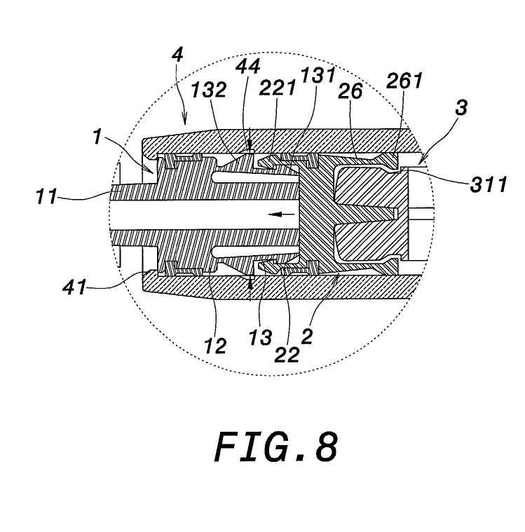

As shown in FIG. 8, the above projective second positioning means 42 can be replaced with a depressed second positioning means 44. Of course, the abutting edges 132 of the passive needle retraction element 1 need to be prolonged accordingly so that the passive needle retraction element 1 can be positioned between the first and second positioning means 41 and 44. The abutting edges 132 are embedded into the depressed second positioning means 44. When the passive needle retraction element 1 is pressed by the active needle retraction element 2, the abutting edges 132 can be separated from limit of the second positioning means 44 by means of contraction of the fastening claws 13.

To sum up, the present invention is characterized in that the dead end of injection stroke is situated at the front end of the barrel 4. After each syringe has been injected, the active and passive needle retraction elements 2 and 1 are engaged together, and the needle 5 is at a state awaiting retraction. At this time, it is only necessary to pull back the push rod 3 to retract the needle 5 into the barrel 4. Furthermore, once the push rod 3 has arrived at the end of needle retraction stroke, its clamped state is immediately released. Therefore, three actions of injection, pulling back, and separation are finished instantaneously. The needle 5 can be retracted into the barrel 4 without any extra operation as compared to the conventional injection.

Although the present invention has been described with reference to the preferred embodiments thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

I claim:

1. A needle retraction type safety syringe comprising:
a passive needle retraction element disposed in a front end of a barrel and engaged with a tail end of a needle, said passive needle retraction element being limited by a second positioning means on an inner wall of said barrel so that it cannot be retracted into said barrel temporarily; and
an active needle retraction element having a resilient and expanding rear section larger than the bore of said barrel so that it can be exactly contracted to clamp an inner end of a push rod when implanted into said barrel, a front section of said active needle retraction element being capable of engaging with said passive needle retraction element and forcing said passive needle retraction element to separate from the positioning restriction of said second positioning means.

2. The needle retraction type safety syringe as claimed in claim 1, wherein the front and rear sections of said active needle retraction elements can clamp said passive needle retraction element and the inner end of said push rod, respectively.

3. The needle retraction type safety syringe as claimed in claim 2, wherein the front and rear sections of said active needle retraction element are respectively composed of a front holding body and a rear holding body, each having a plurality of annularly disposed fastening claws, a rear edge of each of said fastening claws of said rear holding body forming an expansion flange larger than the bore of said barrel, a front edge of each of said fastening claws of said front holding body forming a first fastening body.

4. The needle retraction type safety syringe as claimed in claim 2, wherein a depressed concave edge corresponding to said expansion flange is disposed on an inner wall near a rear end of said barrel so that said active needle retraction element releases its clamping to said push rod when retreating to said depressed concave edge.

5. The needle retraction type safety syringe as claimed in claim 2, wherein said passive needle retraction element comprises a collar and a held body having a plurality of annularly disposed fastening claws, one end of said collar being used to engage a needle, a rear edge of each of said fastening claws of said held body forming a second body, said held body being capable of being sleeved into said active needle retraction element, said first fastening bodies of said active needle retraction element corresponding to said second fastening bodies being used to retain said held body and said active needle retraction element.

6. The needle retraction type safety syringe as claimed in claim 3, wherein said passive needle retraction element comprises a collar and a held body having a plurality of annularly disposed fastening claws, one end of said collar being used to join a needle, a rear edge of each of said fastening claws of said held body forming a second body, said held body being capable of being sleeved into said active needle retraction element, said first fastening bodies of said active needle retraction element corresponding to said second fastening bodies being used to retain said held body and said active needle retraction element.

7. The needle retraction type safety syringe as claimed in claim 1, wherein the rear section of said active needle retraction element has a or a plurality of bosses, and corresponding insertion holes are formed at the inner end of said push rod so that said push rod can be engaged with said active needle retraction element.

8. The needle retraction type safety syringe as claimed in claim 1, wherein the inner wall of the front end of said barrel has a first positioning means and a second positioning means, said first and second positioning means being a plurality of protuberances, opposite inner sides of said protuberances of said first and second positioning means being flat, opposite outer sides of said protuberances of said first and second positioning means being outer slanting edges and inner slanting edges of inclined shape, respectively, said passive needle retraction element being positioned between said first and second positioning means, abutting edges formed at outer edges of said passive needle retraction element abutting against said second positioning means.

9. The needle retraction type safety syringe as claimed in claim 8, wherein said second positioning means can be a region depressed into the inner wall of said barrel so that said abutting edges can be embedded therein.

10. The needle retraction type safety syringe as claimed in claim 8, wherein said protuberances of said first and second positioning means are staggered.
